(19) United States
(12) Patent Application Publication
(10) Pub. No.: US 2022/0356472 A1
(43) Pub. Date: Nov. 10, 2022

(54) METHODS AND COMPOSITIONS FOR TYPE 2 DIABETES THERAPY

(71) Applicant: Sanford Burnham Prebys Medical Discovery Institute, La Jolla, CA (US)

(72) Inventors: Randal J. KAUFMAN, San Diego, CA (US); Jing YONG, San Diego, CA (US)

(21) Appl. No.: 17/774,018

(22) PCT Filed: Nov. 6, 2020
(86) PCT No.: PCT/US20/59364

§ 371 Date: May 3, 2022

Related U.S. Application Data
(60) Provisional application No. 62/933,296, filed on Nov. 8, 2019.

(51) Int. Cl. C12N 15/113; A61P 1/16; A61K 31/138; C12N 15/86

(52) U.S. Cl. CPC C12N 15/113; A61P 1/16; A61K 31/138; C12N 15/86; C12N 2320/32; C12N 2310/3513; C12N 2310/11; C12N 2310/14; C12N 2840/002; C12N 2740/10045; C12N 2740/10043

(57) ABSTRACT

Methods and compositions related to type 2 diabetes therapy are described, where the methods and compositions relieve ER stress in pancreatic islet cells. The present disclosure provides methods and compositions for reducing hepatic steatosis, e.g., associated with Type 2 diabetes (T2D). In one aspect, the present disclosure provides a method of reducing hepatic steatosis, e.g., associated with T2D, in a subject in need thereof.

### CROSS REFERENCE

This application claims the benefit of U.S. Provisional Application No. 62/933,296, filed on Nov. 8, 2019; which is incorporated herein by reference in its entirety.

### BACKGROUND

Type 2 diabetes (T2D) is a metabolic disorder that poses a severe health challenge for modern society as it is estimated by the United States' Centers for Disease Control and Prevention that thirty million Americans are affected by this condition (Prevention, 2017). People with T2D are more likely to develop other serious cardiovascular and liver diseases. T2D is characterized by insulin resistance (IR), hyperglycemia and hyperinsulinemia (Weyer et al., 2001). Hyperinsulinemia is the driving force for insulin resistance. In the early phase of T2D insulin synthesis and secretion by pancreatic β cells is enhanced. Enhanced insulin synthesis is associated with proinsulin misfolding and endoplasmic reticulum (ER) stress.

Current T2D therapeutics focus on achieving improved blood glucose control by improving both insulin secretion and reducing peripheral IR. However, newer avenues for therapeutic intervention are sought to reduce the disease burden.

### SUMMARY OF THE DISCLOSURE

The present disclosure provides methods and compositions for reducing hepatic steatosis, e.g., associated with Type 2 diabetes (T2D). In one aspect, the present disclosure provides a method of reducing hepatic steatosis, e.g., associated with T2D, in a subject in need thereof, comprising: selectively inhibiting C/EBP homologous protein (Chop) in pancreatic β cells, e.g., by administering to the subject a composition comprising: (a) a Chop inhibiting moiety, and (b) a pancreatic β cell targeting moiety. In some embodiments, the method can be used for treating T2D. In some embodiments, the method for reducing hepatic steatosis and/or treating T2D comprises inhibiting Chop selectively in pancreatic β cells. In some embodiments, the method comprises treating hepatic steatosis. In some embodiments, the method comprises reducing ER stress of pancreatic β cells. In some embodiments, the subject is a mammal. In some embodiments, the subject is a human. In some embodiments, the instant disclosure provides methods and compositions that may prevent aging associated non-alcoholic fatty liver disease (NAFLD), or at least a symptom or a manifestation associated thereof.

In another aspect, the present disclosure provides a method of regulating C/EBP homologous protein (Chop) in pancreatic β cells. In some embodiments, the method comprises administering a nucleic acid composition comprising: (a) a Chop inhibiting moiety, and (b) a targeting moiety that directs the Chop inhibiting moiety to a target in a pancreatic cell. In some embodiments, regulating C/EBP homologous protein (Chop) in pancreatic β cells ameliorates or treats one or more diseases. In some embodiments, regulating C/EBP homologous protein (Chop) in pancreatic β cells ameliorates or treats one or more diseases associated with fatty liver disease. In some embodiments, regulating C/EBP homologous protein (Chop) in pancreatic β cells ameliorates or treats a chronic disease, including diseases such as cardiovascular disease (CVD), chronic kidney disease (CKD), colorectal carcinoma, type 2 diabetes mellitus (T2DM), and psoriasis. In some embodiments, regulating C/EBP homologous protein (Chop) in pancreatic β cells ameliorates or treats one or more conditions for which fatty liver disease is a risk factor, including conditions such as colonic neoplasia and reduced bone mineral density.

In some embodiments, a Chop inhibiting moiety is a nucleic acid. In some embodiments the Chop inhibiting moiety and the pancreatic β cell targeting moiety are operably linked. In some embodiments, the nucleic acid is an RNA. In some embodiments, the nucleic acid is an inhibitory RNA. In some embodiments, the nucleic acid is an antisense oligomeric RNA. In some embodiments, the nucleic acid is an iRNA.

In some embodiments, the pancreatic β cell targeting moiety is a peptide. In some embodiments, the peptide is internalized by a pancreatic cell. In some embodiments, the peptide is glucagon-like peptide 1 (GLP-1), or a fragment thereof.

In some embodiments, the Chop inhibiting moiety is a nucleic acid editing moiety. In some embodiments, the nucleic acid editing moiety is a genomic DNA editing moiety. In some embodiments, the nucleic acid editing moiety comprises a nuclease. In some embodiments, the nucleic acid editing moiety comprises a recombinase. In some embodiments, the pancreatic β cell targeting moiety comprises a guiding nucleic acid sequence.

In some embodiments, the Chop inhibiting moiety and/or the pancreatic β cell targeting moiety is inducible by an inducer. In some embodiments, the inducer can be administered ex vivo. In some embodiments, the targeting moiety is inducible by an inducer. In some embodiments, the inducer is administered ex vivo. In some embodiments, the inducer is tamoxifen.

In some embodiments, wherein the subject is a mammal. In some embodiments, the subject is a human. In some embodiments, the subject is a non-human mammal. In some embodiments the administering comprises administering to the subject systemically. In some embodiments, the administering reduces or alleviates pancreatic β cell stress. In some embodiments, the administering reduces total pancreatic insulin content.

In one aspect, the present disclosure provides a nucleic acid composition comprising a nucleic acid sequence capable of suppressing a Chop gene expression, operably linked to a peptide. In some embodiments, the peptide is conjugated to the nucleic acid sequence. In some embodiments, the nucleic acid composition comprises a nucleic acid construct comprising: (a) an antisense oligomeric (ASO) sequence, and (b) a GLP-1 peptide or a fragment thereof. In some embodiments, the nucleic acid construct further comprises a linker.

In some embodiments, the nucleic acid sequence capable of suppressing a Chop expression is an RNA. In some embodiments, the RNA is an inhibitory RNA. In some embodiments, the RNA is an antisense oligomeric RNA. In some embodiments, the RNA is an iRNA. In some embodiments, nucleic acid composition is targetable to a pancreatic cell. In some embodiments, nucleic acid composition is targetable to a pancreatic β cell.

In some embodiments, the peptide is glucagon-like peptide 1 (GLP-1), or a fragment thereof.

In some embodiments, the nucleic acid composition further comprises a linker. In some embodiments, the linker is a synthetic linker. In some embodiments, the linker may be a chemical linker. In some embodiments, the linker may be a short peptide linker. In some embodiments, the linker physically connects the antisense oligomeric sequence, and the GLP-1 peptide.

The linker can be a chemical linker. The linker can be a synthetic linker. The linker may be able to crosslink the nucleic acid sequence and the peptide.

In some embodiments, the nucleic acid composition comprises a nucleic acid sequence-a linker-a GLP1 peptide.

In some embodiments the nucleic acid composition further comprises a delivery vehicle. In some embodiments, the delivery vehicle comprises a lipid component. In some embodiment the lipid is in the form of a liposome. In some embodiments, the delivery vehicle comprises a lipid, such as a cationic lipid. In some embodiments, the delivery vehicle comprises or a vector, such as a viral vector or a nucleic acid construct comprising the nucleic acid sequence. In some embodiments, the nucleic acid composition comprises a vector.

In some embodiments the present disclosure provides a cell comprising a composition described herein, or a part thereof.

In some embodiments, the nucleic acid composition can be used for preparing a therapeutic for treating diabetes. In some embodiments, the nucleic acid composition that can be used for preparing the therapeutic for treating diabetes is inside a cell. In some embodiments the cell is a pancreatic β cell.

In some embodiments, the present disclosure provides a pharmaceutical composition, comprising the nucleic acid described herein or a part thereof, and a pharmaceutically acceptable carrier. The nucleic acid may be comprised in a cell. In some embodiments, the nucleic acid described herein, or a part thereof may be comprised in a vector. In some embodiments, the pharmaceutical composition, comprising the nucleic acid comprises the vector.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which.

FIG. 1 is a schematic diagram showing a plausible mechanism of action of Chop-mediated greater insulin production in response to unfolded protein response (UPR).

FIG. 2A is a schematic diagram of a loxP construct for disrupting endogenous Chop exon 3.

FIG. 2B is a diagram showing an example of a sequencing reaction, where the sequences comprise loxP left and right arm sequences and flanking sequences at the site of successful integration.

FIG. 3A shows an exemplary data depicting changes in body weight of tamoxifen inducible floxed mice, that is, Chop+/+:Cre and Chop Fe/Fe: Cre mice.

FIG. 3B shows an exemplary data depicting changes in blood glucose of tamoxifen inducible floxed mice, that is, Chop+/+: Cre and Chop Fe/Fe: Cre mice.

FIG. 3C shows an exemplary data depicting comparison of pancreatic insulin levels in cell-specific Chop deleted (Chop^{Δ/Δ}:Cre) with Chop+/+: Cre mice.

FIG. 3D shows an exemplary data depicting correlation of pancreatic insulin levels with liver weight in β cell-specific Chop deleted (Chop^{Δ/Δ}:Cre, red triangles) with Chop+/+: Cre (black triangles) mice.

FIG. 3E shows an exemplary data depicting correlation of pancreatic insulin levels with liver triglycerides (TG) in β cell-specific Chop deleted (Chop^{Δ/Δ}:Cre, red squares) and Chop+/+: Cre (black squares) mice.

FIG. 3F shows an exemplary photographic data depicting liver morphology in Chop+/+, β cell-specific Chop deleted, and Chop heterogenous (+/Δ) mice.

FIG. 3G shows exemplary data for liver weight in wild-type (Chop+/+: Cre) and Chop βKO mice (Chop Δ/Δ: Cre).

FIG. 3H shows exemplary data for liver triglyceride levels in wild-type (Chop+/+: Cre) and Chop βKO mice (Chop Δ/Δ: Cre).

FIG. 4A shows a schematic flow diagram of hyperglycemic clamp experiment in mice. HFD, high fructose diet.

FIG. 4B-4D depicts experimental results of hyperglycemic clamp on arterial insulin, arterial glucose and arterial C-peptide respectively in Chop+/−: Cre and Chop+/Δ: Cre mice.

FIG. 4E depicts experimental results indicating that ChopKO mice require significantly higher glucose infusion to maintain blood glucose levels. GIR, glucose infusion rate.

FIG. 4F-4G depicts experimental results indicating no significant difference in insulin immunoreactive areas in in Chop+/−: Cre and Chop+/Δ: Cre mice.

FIG. 5A depicts experimental results indicating that both Ins1 and Ins2 transcripts were decreased in Chop βKO islets by about 75%.

FIG. 5B depicts experimental results indicating successful reduction in Chop transcript, and reduction in UPR transcripts in Chop βKO islets.

FIG. 5C shows an exemplary volcano plot representing transcriptome profiling, indicating significant reduction in UPR gene expressions in Chop βKO islets, represented for example by Atf3, Bip/Hspa5 and Dnajb9/ERdj4 transcripts.

FIG. 5D shows exemplary qPCR analysis results depicting transcripts that are associated with the pancreatic β cell characteristics, i.e., identity were not altered.

FIG. 5E shows exemplary qPCR analysis results depicting transcripts that are downstream of IRE1α, the PERK and the ATF6α branches of the UPR, respectively, were reduced.

FIG. 5F shows a heat map plot representing data depicting exemplary gene expression in the two types of mice as indicated.

FIG. 5G shows an immunoblot data resulting from pulldown with anti-insulin antibody in the islets of two types of mice as indicated.

FIG. 6A shows a set of experimental results depicting exemplary gene expression of the insulin panel genes, including insulin 1, and 2, following treatment of isolated islet cells from WT C57Bl6 mice with 10 μM BAPTA-AM.

FIG. 6B shows experimental results indicating exemplary calcium flux in control and Chop β-knockout C57Bl6 mice.

FIG. 7A shows GLP1-ASO structure, showing a Chop-antisense oligonucleotide (ASO) conjugated to a GLP-1 via a linker.

FIG. 7B shows data indicating GLP1-Chop-ASO reduces Chop mRNA (expressed as TPM) specifically in murine islets compared to liver tissue. RNA extracted from the livers of the same mice served as internal controls for Chop expression, for RNA-Seq. Expression of additional C/EBP transcription factor family members are shown as controls.

FIG. 7C shows data indicating GLP1-Chop-ASO had no obvious effects on glucose-induced cytosolic Ca2+ level, and reduced ER Ca2+ pools in primary islets in vivo. Representative intracellular Ca2+ traces (mean±SEM) are shown (n=30-50 islets/group). Male and female Chop foxed mice (with no RIP-CreER gene) were injected I.P. with control or GLP1-Chop-ASOs 8 days before islet isolation. Two-way ANOVA with post-hoc Tukey multiple comparison test was used for statistical analysis.

FIG. 8A shows hematoxylin and eosin (H & E) stained liver tissue showing onset of NAFLD as evidenced by enlarged hepatocytes size by H&E staining in mice with age, sacrificed at 11-14 and 17 months of age that are fed normal diet. Chop gene knocked out mice (Chop-KO mice) prevented age-related NAFLD as evidenced by normal-sized hepatocytes.

FIG. 8B shows experimental results indicating increased liver weight expressed as percentage of body weight (p<0.05 by 2-way ANOVA from “Age”); and pancreatic β-cell specific Chop gene KO leads to a reduction in fasting serum insulin concentrations that correlate with NAFLD prevention in aged mice as reflected by decreased liver weight expressed as percentage of body weight (p<0.05 by 2-way ANOVA from “Chop”) comparing to their littermates control group.

FIG. 8C shows data indicating triglyceride levels in WT and Chop gene KO mice liver that correlate with other data indicating Chop deletion prevents NAFLD.

FIG. 9A shows data indicating results from in vitro glucose-stimulated insulin secretion (GSIS) assay along with metabolic pulse-chase labeling of nascent proinsulin (ProIns), with ³⁵S-Methione+³⁵S-Cysteine in order to track the intracellular ProIns processing at the indicated time period.

FIG. 9B shows scintillation counts for ³⁵S-Methione/-Cysteine in the experiment of FIG. 9A.

FIG. 9C shows data indicating cumulative insulin secretion into the condition media measured by ELISA assay: while there is a discernible reduction in insulin release from Chop-null islets at 1 hr post 16.7 mM glucose stimulation.

FIG. 10A shows cytological TUNEL assay, tissue section staining of Chop FE/FE: Cre and Chop Δ/Δ (Chop deleted) mice pancreas for apoptosis detection.

FIG. 10B shows graph indicating the number of islets surveyed per pancreatic tissue section.

FIG. 10C shows number of TUNEL positive nuclei per pancreatic tissue section.

FIG. 10D shows number of Ki67 positive nuclei per pancreatic tissue section.

### DETAILED DESCRIPTION

The present disclosure is based on the finding that alleviating ER stress in β cells while maintaining optimal insulin secretion can be possible in living systems which may reduce or alleviate one or more aspects of T2D and T2D associated diseases such as fatty liver or hepatic steatosis.

Echoing clinical findings made in the Pima Indians (Weyer et al., 2000), it was discovered that hyperinsulinemia serves as the driving force for insulin resistance in mouse T2D models (Mehran et al., 2012; Templeman et al., 2015). In the Mehran and Johnson paradigm (Mehran et al., 2012), excessive amounts of insulin from pancreatic β cell is considered to causal for peripheral IR. However, observations using Insulin gene KO models are of uncertain relevance to human T2D. A practical molecular target along with corresponding pharmacological interventions are critically needed to evaluate whether a reduction in insulin dosage can achieve the same effect observed in mouse models.

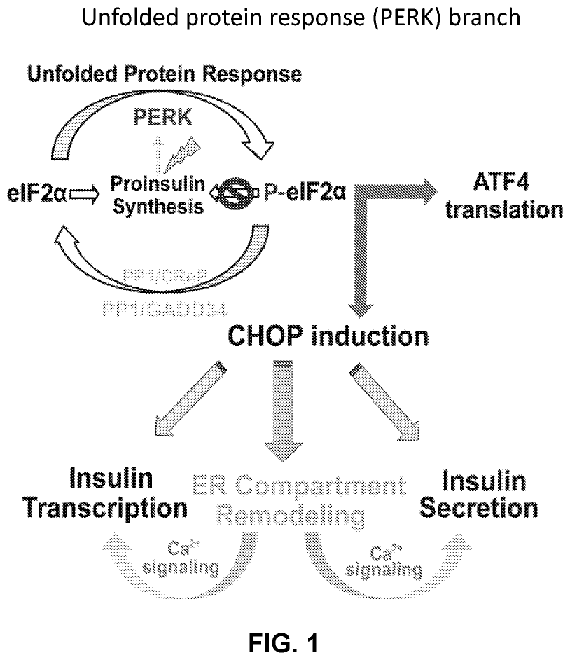

Presented herein are experimental results indicating new and exciting findings leading to the notion that unfolded protein response (UPR) can mediate Chop induction, which may be related to excessive amounts of insulin from pancreatic β cells, which in turn can be related to hepatic steatosis or fatty liver (FIG. 1). At a molecular level, unfolded protein response (UPR) may be activated upon the accumulation of misfolded proteins in the endoplasmic reticulum (ER), that may be sensed by the binding immunoglobulin protein (BiP)/glucose-regulated protein 78 (GRP78). The accumulation of unfolded proteins may sequester BiP so it can dissociate from three ER-transmembrane transducers, which may lead to their activation. These transducers are inositol requiring (IRE) 1α, PKR-like ER kinase (PERK) and activating transcription factor (ATF) 6α. PERK can phosphorylate eukaryotic initiation factor 2 alpha (eIF2α) resulting in global mRNA translation attenuation, and concurrently can selectively increase the translation of several mRNAs, including the transcription factor ATF4, and its downstream target CHOP. IRE1α has kinase and endoribonuclease (RNase) activities. IRE1α may undergo autophosphorylation, which may activate the RNase activity to cleave XBP1 mRNA, to produce the active transcription factor sXBP1. IRE1α activation may also recruit and activate the stress kinase JNK. ATF6α may transit to the Golgi compartment where it can be cleaved by intramembrane proteolysis to generate a soluble active transcription factor.

UPR pathways can act in concert to increase ER content, expand the ER protein folding capacity, degrade misfolded proteins, and can reduce the load of new proteins entering the ER. All of these are geared toward adaptation to resolve the protein folding defect. Faced with persistent ER stress, adaptation can start to fail and apoptosis may occur, possibly mediated through calcium perturbations, reactive oxygen species, and the proapoptotic transcription factor CHOP. UPR is activated in several liver diseases; including obesity associated fatty liver disease, viral hepatitis, NAFLD and alcohol-induced liver injury, all of which are associated with steatosis, raising the possibility that ER stress-dependent alteration in lipid homeostasis is the mechanism that underlies the steatosis.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

The term “about” or “approximately” may mean within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the present disclosure, and vice versa. Furthermore, compositions of the present disclosure may be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Reference in the specification to a “cell” may refer to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell may be a synthetically made, sometimes termed an artificial cell).

Reference in the specification to “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g. deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled by well-known techniques. Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,NcN′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides may include [R6G]dUTP, [TAMRA]dUTP, [R110]dCTP, [R6G]dCTP, [TAM RA]dCTP, [JOE]ddATP, [R6G]ddATP, [FAM]ddCTP, [R110]ddCTP, [TAN1RA]ddGTP, [ROX]ddTTP, [dR6G]ddATP, [dR110]ddCTP, [dTAM RA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, TR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-1 4-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides may also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-cICTP, biotin-14-dCTP), and biotin-dUTP (e.g. biotin-11-dUTP, biotin-1.6-dUTP, biotin-20-dUTP).

Terms such as “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide may comprise one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, eDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

Reference in the specification to “conjugated” may be used to designated chemically bonded i.e., attached by chemical bonds. A conjugate is a molecule, example a peptide that is chemically (for example covalently) linked to a biomolecule or molecule of interest, for example, a nucleic acid, that is conjugated to another molecule.

Reference in the specification to “operably linked” refers to a functional relationship between two or more nucleic acid segments. Typically, it refers to the functional relationship of a transcriptional regulatory sequence to a transcribed sequence.

“Polyadenylation sequence” (also referred to as a “poly A⁺ site” or “poly A⁺ sequence”) refers to a DNA sequence which directs both the termination and polyadenylation of the nascent RNA transcript. Efficient polyadenylation of the recombinant transcript is desirable, as transcripts lacking a poly A⁺ tail are typically unstable and rapidly degraded. The poly A⁺ signal utilized in an expression vector may be “heterologous” or “endogenous”. An endogenous poly A⁺ signal is one that is found naturally at the 3′ end of the coding region of a given gene in the genome. A heterologous poly A⁺ signal is one which is isolated from one gene and placed 3′ of another gene, e.g., coding sequence for a protein. A commonly used heterologous poly A⁺ signal is the SV40 poly A⁺ signal. The SV40 poly A⁺ signal is contained on a 237 bp BamHI/BclI restriction fragment and directs both termination and polyadenylation; numerous vectors contain the SV40 poly A⁺ signal. Another commonly used heterologous poly A⁺ signal is derived from the bovine growth hormone (BGH) gene; the BGH poly A⁺ signal is also available on a number of commercially available vectors. The poly A⁺ signal from the Herpes simplex virus thymidine kinase (HSV tk) gene is also used as a poly A⁺ signal on a number of commercial expression vectors. The polyadenylation signal facilitates the transportation of the RNA from within the cell nucleus into the cytosol as well as increases cellular half-life of such an RNA. The polyadenylation signal is present at the 3′-end of an mRNA.

Reference in the specification to “exon” refers to a nucleic acid sequence found in genomic DNA that is bioinformatically predicted and/or experimentally confirmed to contribute contiguous sequence to a mature mRNA transcript.

Reference in the specification to “intron” refers to a sequence present in genomic DNA that is bioinformatically predicted and/or experimentally confirmed to not encode part of or all of an expressed protein, and which, in endogenous conditions, is transcribed into RNA (e.g. pre-mRNA) molecules, but which is spliced out of the endogenous RNA (e.g. the pre-mRNA) before the RNA is translated into a protein.

Reference in the specification to “complement,” “complements,” “complementary,” and “complementarity,” as used herein, can refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/embossneedle/nucleotide.html), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.ukaools/psa/emboss_water/nucleotide.html, optionally with default settings). Optimal alignment can be assessed using any suitable parameters of a chosen algorithm, including default parameters. Complementarity may be perfect or substantial/sufficient. Perfect complementarity between two nucleic acids may mean that the two nucleic acids may form a duplex in which every base in the duplex is bonded to a complementary base by Watson-Crick pairing. Substantial or sufficient complementary may mean that, a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions may be predicted by using the sequences and standard mathematical calculations to predict the melting temperature (Tₘ) of hybridized strands, or by empirical determination of Tₘ by using routine methods.

The term “knockout” (“KO”) or “knocking out” as used herein refers to a deletion, deactivation, or ablation of a gene in a cell, or in an organism, such as, in a pig or other animal or any cells in the pig or other animal. KO, as used herein, may also refer to a method of performing, or having performed, a deletion, deactivation or ablation of a gene or portion thereof, such that the protein encoded by the gene is no longer formed.

The term “knockin” (“KI”) or “knocking in” as used herein refers to an addition, replacement, or mutation of nucleotide(s) of a gene in a pig or other animal or any cells in the pig or other animal. KI, as used herein, may also refer to a method of performing, or having performed, an addition, replacement, or mutation of nucleotide(s) of a gene or portion thereof.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Reference in the specification to “derivative,” “variant,” and “fragment,” may be with regards to a polypeptide, can indicate a polypeptide related to a wild type polypeptide, for example either by amino acid sequence, structure (e.g., secondary and/or tertiary), activity (e.g., enzymatic activity) and/or function. Derivatives, variants and fragments of a polypeptide may comprise one or more amino acid variations (e.g., mutations, insertions, and deletions), truncations, modifications, or combinations thereof compared to a wild type polypeptide.

Reference in the specification to “percent (%) identity,” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps may be introduced in one or both of the candidate and reference sequences for optimal alignment and non-homologous sequences may be disregarded for comparison purposes). Alignment, for purposes of determining percent identity, may be achieved in various ways that are within the skill in the art, for instance, using publicly available computer software such as BLAST, ALIGN, or Megalign (DNASTAR) software. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

Reference in the specification to “nucleic acid editing moiety,” can indicate a moiety, which may induce one or more gene edits in a polynucleotide sequence. The polynucleotide sequence may be in a host cell. Alternatively, the polynucleotide sequence may not be in a host cell. Gene editing using the nucleic acid editing moiety may comprise introducing one or more heterologous polynucleic acids (for example, genes, or fragments thereof) in a cell, or deleting one or more endogenous polynucleic acids (for example, genes, or fragments thereof) from the cell. In some cases, gene editing using the nucleic acid editing moiety may comprise substituting any one or more polynucleic acids (for example, genes, or fragments thereof) thereof. In some cases, gene editing using the nucleic acid editing moiety may comprise a combination of any of the above, either simultaneously or sequentially. In some cases, the one or more polynucleic acids may be a DNA. In some cases, the one or more polynucleic acids may be genomic DNA. In some cases, the any one or more genes or nucleic acid portions thereof may be added to or deleted from the chromosomal DNA of a cell by the nucleic acid editing moiety. In some cases, the one or more polynucleic acids may be genomic DNA. In some cases, one or more polynucleic acids may be added to or deleted from the chromosomal DNA of a cell by the nucleic acid editing moiety, that is not part of a gene. In some cases, the one or more polynucleic acids may be contained in exosomes. In some cases, one or more polynucleic acids may be in mitochondria or any other cell organelle. In some cases, the any one or more genes or nucleic acid portions thereof may be added to or deleted from the episomal DNA or epichromosomal DNA of the cell by the nucleic acid editing moiety. In some cases, one or more polynucleic acids may be RNA. In some cases, one or more exogenous polynucleic acids may be added into the genomic DNA, via integration of the exogenous polynucleic acids into the genomic DNA. Integration of any one or more genes into the genome of a cell may be done using any suitable method. Non-limiting examples of suitable methods for the genomic integration and/or genomic replacement strategies disclosed and described herein include CRISPR-mediated genetic modification using Cas9, Cas12a (Cpf1), or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof. Designer zinc fingers, transcription activator-like effectors (TALEs), or homing meganucleases are available for producing targeted genome perturbations.

Targeted genome editing is possible via CRISPR-mediated genetic modification using a Cas or Cas-like endonuclease. CRISPR (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli, and associated genes. Similar interspersed SSRs may be identified in Haloferax mediterranei, Streptococcus pyogenes, Anabaena, and Mycobacterium tuberculosis. The CRISPR loci typically differ from other SSRs by the structure of the repeats, which have been termed short regularly spaced repeats (SRSRs). The repeats are short elements that occur in clusters that are regularly spaced by unique intervening sequences with a substantially constant length. Although the repeat sequences are highly conserved between strains, the number of interspersed repeats and the sequences of the spacer regions typically differ from strain to strain. CRISPR loci have been identified in more than 40 prokaryotes including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

Cas9 gene may be found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region. A Cas 9 protein may be from an organism from a genus comprising, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, or Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus.

The nucleic acid editing moiety may comprise a nucleic acid cleavage moiety. The nucleic acid cleavage moiety may introduce a break or a cleavage in a nucleic acid site molecule. The nucleic acid cleavage moiety may be capable of recognizing a specific cleavage recognition site, for example, when in proximity to the cleavage recognition site on a target polynucleotide sequence. In some cases, the nucleic acid cleavage moiety may be directed by a second molecule (such as a nucleic acid, e.g. sequence specific guide RNA for Cas9) to a specific cleavage site on a polynucleic acid, for introducing a break or cleavage on the polynucleic acid. The nucleic acid cleavage moiety may initiate an introduction, deletion or substitution of the nucleic acid in the genomic DNA. In some cases, the nucleic acid cleavage moiety is a nuclease, or a functional fragment thereof. In some cases, the nucleic acid cleavage moiety may comprise an endonuclease, an exonuclease, a DNase, an RNase, a strand-specific nuclease, or a more specialized nuclease, (for example, a CRISPR associated protein 9, Cas 9), or any fragment thereof. In some cases, the nucleic acid cleavage moiety may be nickase.

In some cases, the nuclease is an AAV Rep protein, Rep68/78.

In some cases, a nucleic acid editing moiety may comprise a viral machinery or a fragment thereof that is capable of incorporating a viral gene into a host cell. For example, a nucleic acid editing moiety may refer to a viral integrase system, such as a lentiviral integrase system. Integrase is a retroviral enzyme that catalyzes integration of DNA into the genome of a mammalian cell, a useful step of retrovirus replication in the retroviral infection process. The process of integration can be divided into two sequential reactions. The first one, named 3′-processing, corresponds to a specific endonucleolytic reaction which prepares the viral DNA extremities to be competent for the subsequent covalent insertion, named strand transfer, into the host cell genome by a trans-esterification reaction. In some cases, a nucleic acid editing moiety may additionally refer to a transposon/transposase or a retrotransposase system or a component thereof, for integration of a piece of DNA into the genome. However, inserting exogenous DNA into specific genomic sequences is preferred over random and semi-random integration throughout the target cell's genome, such as with some retroviral vectors and transposons/transposases. The random and semi-random integration procedures may result in outcomes such as positional-effect variegation, transgene silencing, and, in some cases, insertional mutagenesis caused by transcriptional deregulation or physical disruption of endogenous target-cell genes.

Reference in the specification to antisense oligomeric nucleic acids or antisense oligonucleotides or ASOs refers to antisense RNA, that can be synthetic single-stranded deoxyribonucleotide analogs, usually 15-30 bp in length. In some embodiments, the antisense RNA is 10-50 nucleotides in length. In some embodiments, the antisense RNA is 15-45 nucleotides in length. In some embodiments, the antisense RNA is 20-50 nucleotides in length. In some embodiments, the antisense RNA is 20-40 nucleotides in length. In some embodiments, the antisense RNA is 15-40 nucleotides in length. In some embodiments, the antisense RNA is 10-30 nucleotides in length. In some embodiments, the antisense RNA is 20-30 nucleotides in length. In some embodiments, the antisense RNA is 22-30 nucleotides in length. In some embodiments, the antisense RNA is 20-27 nucleotides in length. In some embodiments, the antisense RNA is 21-27 nucleotides in length. In some embodiments, the oligomeric nucleic acids is single stranded. In some embodiments, the oligomeric nucleic acids sequence (3′ to 5′) is antisense and complementary to the sense sequence of the target nucleotide sequence. In some embodiments, the oligomeric nucleic acids sequence is a double stranded ribonucleic acid, e.g., an siRNA, also called an iRNA. In some embodiments, the antisense oligomer comprises modified and/or unmodified nucleotides. In some embodiments, unmodified oligonucleotides after quick degradation by circulating nucleases are excreted by the kidney; unmodified oligonucleotides are generally too unstable for therapeutic use. Therefore, chemical modification strategies have been developed to overcome this and other obstacles in ASO therapy program. Commonly used modification in these ASOs is 2′ ribose modifications that include 2′-O-methoxy (OMe), 2′-O-methoxy-ethyl (MOE), and locked nucleic acid (LNA). 2′-OMe modifications are commonly used in a ‘gapmer’ design, which is a chimeric oligo comprising a DNA sequence core with flanking 2′-MOE nucleotides that enhances the nuclease resistance, in addition to lowering toxicity and increasing hybridization affinities. Sequence specific “small inhibiting RNA (siRNA)” or “iRNA” relates to small RNA sequences that bind to a target nucleic acid molecule, which can expression of a gene expression product. Introduction of double-stranded RNA (dsRNA) also called interfering RNA (RNAi), or hairpin RNA is an effective trigger for the induction of gene silencing in a large number of eukaryotic organisms, including animals, fungi, and plants. Both the qualitative level of dsRNA-mediated gene silencing (i.e., the level of gene silencing within an organism) and the quantitative level (i.e., the number of organisms showing a significant level of gene silencing within a population) have proven superior to the more conventional antisense RNA or sense RNA mediated gene silencing methods. In some embodiments, the antisense oligonucleotide (ASO) comprises at least 5 consecutive nucleotides that are complementary to and antisense of a nucleic acid sequence encoding a human protein, such as a C/EBP homologous protein. In some embodiments, the antisense oligonucleotide (ASO) comprises at least 6, 7, 8, 9, 10, 11, 12, 13, 14, 15, 16, 17, 18, 19, 20, 21, 22, 23, 24, 25, 26, 27, 28, 29, or at least 30 consecutive nucleotides that are complementary and antisense of a nucleic acid sequence encoding a human C/EBP homologous protein. In some embodiments, the ASO is an siRNA, having 21-27 nucleotide pairs, and at least one nucleotide overhang at the 5′ and the 3′ end.

In some embodiments the ASO is a single stranded oligomer. In some embodiments the single stranded oligomer comprises 20-50 nucleotides in length. In some embodiments the single stranded oligomer comprises 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 nucleotides.

Another method of inhibiting gene expression comprises targeting a nucleic acid molecule to an anti-sense transcript and sense strand transcript, wherein the nucleic acid molecule targeting the anti-sense transcript is complementary to the anti-sense strand and the nucleic acid molecule targeting the sense transcript is complementary to the sense strand; and, binding of the nucleic acid to the anti-sense and sense transcript, thereby, inhibiting gene expression. The nucleic acid molecule is a RNA molecule and the nucleic acid molecules targeting the anti-sense and sense transcripts bind said transcripts in convergent, divergent orientations with respect to each other and/or are overlapping. Method for gene suppression in eukaryotes by transformation with a recombinant construct containing an antisense and/or sense nucleotide sequence for the gene(s) to be suppressed is known in the art.

The present disclosure provides methods and compositions for targeting Chop expression, which may be associated with insulin secretion in ER stress, downstream of unfolded protein response.

In one aspect pancreatic β cell Chop may be targeted. In the aspect, provided herein is a method of inhibiting C/EBP homologous protein (Chop) in pancreatic β cells by administering to the subject a composition comprising: (a) a Chop inhibiting moiety, and (b) a pancreatic β cell targeting moiety. A pancreatic β cell Chop may be inhibited by targeted deletion of pancreatic β cell Chop. This may be achieved by any method, including but not limiting to using site specific nucleases and recombinases to direct and delete Chop in pancreatic beta cells using transposons, retroposons, TALENs, zinc finger proteins, CRISPR-Cas systems or Cre-lox systems or viral integrase systems.

In some embodiments, the CHOP inhibiting moiety is a nucleic acid. In some embodiments, the CHOP inhibiting moiety is a DNA. In some embodiments, the CHOP inhibiting moiety is an RNA. In some embodiments, the CHOP inhibiting moiety is an inhibitory RNA. In some embodiments, the RNA is an antisense oligomeric nucleic acid, also called antisense oligonucleotide or ASO. In some embodiments, the RNA is a double stranded molecule capable of inhibiting CHOP expression. In some embodiments the RNA is a single stranded structure capable of inhibiting CHOP expression. In some embodiments, the RNA is an antisense oligomeric nucleic acid capable of reducing or inhibiting the expression of pancreatic β cell Chop mRNA. In some embodiments the ASO is about 10-1000 nucleotides long. In some embodiments the nucleic acid is 10-500 nucleotides long, 10-400 nucleotides long, 10-300 nucleotides long, 10-200 nucleotides long, or 10-100 nucleotides long or any length in between. In some embodiments the ASO is about 20, 25, 30, 35, 40, 45, 50, 55, 60, 65, 70, 75, 80, 85, 90, 95, 100, 105, 110, 120, 130, 140, 150, 160, 170, 180, 190, 200, 205, 210, 215, 220, 225, 230, 235, 240, 245, 250, 255, 260, 265, 270, 275, 280, 285, 290, 295 or about 300 nucleotides long. In some embodiments the ASO is less than 100 nucleotides long, less than 90, or less than 80, or less than 70, or less than 60, or less than 50, or less than 40, or less than 30 nucleotides long or any length in between. In some embodiments the nucleic acid is 10-50 nucleotides long, or 12-45, or 15-30 nucleotides long.

In some embodiments, the CHOP inhibiting moiety comprises a nucleic acid sequence that has about at least 80% sequence identity to a contiguous stretch of nucleotides of the length of the ASO within an mRNA encoding CHOP. In some embodiments, the ASO comprises a nucleic acid sequence that that has about at least 80% sequence identity to a contiguous stretch of nucleotides of the length of the ASO within an mRNA encoding a human CHOP. In some embodiments, the inhibitory RNA has a sequence homology or complementarity with at least 10, at least 12, at least 14, at least 16, at least 18, at least 20, at least 25, at least 30, at least 35, at least 40, at least 45 or at least 50 contiguous nucleotides of the CHOP gene sequence or the CHOP mRNA.

In some embodiments, the CHOP inhibiting moiety is a single stranded RNA.

In some embodiments, the CHOP inhibiting moiety is a morpholino antisense oligomer.

In some embodiments, the CHOP inhibiting moiety is a double stranded RNA.

In some embodiments, the CHOP inhibiting moiety comprises an RNA that comprises at least one or more modified bases. In some embodiments, the modified base is a pseudouridine, or 2-methyl cytosine. In some embodiments, the RNA may be stabilized, by one or more of i) substituting at least one naturally occurring nucleotide base with a modified base, ii) conjugating with a biomolecule; such as a peptide.

In some embodiments the pancreatic β cell targeting moiety is a protein, peptide or nucleic acid that can direct the Chop inhibiting moiety to the pancreatic cell. In some embodiments the pancreatic β cell targeting moiety is an antibody that can be conjugated to the chop inhibiting moiety. In some embodiments the pancreatic β cell targeting moiety is a peptide. The peptide may be conjugated to the antisense oligomer. In some embodiments, the peptide is conjugated to a linker which links the peptide at one end and the oligonucleotide in the other. The linker may be a chemical crosslinker. There are several synthetic or chemical cross-linkers.

In some embodiments, the peptide stabilizes the ASO. In some embodiments, the nucleic acid is targeted to a particular cell by the peptide. In some embodiments, the targeting moiety is a molecule capable of binding to a receptor on pancreatic β cell and be internalized into the cell. In some embodiments, the peptide is a GLP-1 peptide, or a fragment thereof. In some embodiments, the peptide comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids.

Alternatively, in some embodiments, the CHOP inhibiting moiety is a DNA sequence. The DNA sequence may comprise a modified nucleotide. The DNA sequence may encode one or more protein of interest, which when expressed, can inhibit the expression of Chop in a pancreatic β cell. In some embodiments, the DNA may comprise a sequence encoding a nuclease, an integrase, or a recombinase.

Alternatively, the DNA may comprise a sequence, which can be inserted into a certain locus of a target cell by a targeting moiety. The targeting moiety in this case may be a nuclease, an integrase, or a recombinase. The DNA may comprise a nucleic acid sequence, which may be introduced within a gene or a chromosomal locus by the action of the targeting moiety, can inhibit the expression of Chop in a pancreatic β cell.

In one aspect, the targeting of the pancreatic β cell Chop may be regulated. Provided herein is a method of regulating pancreatic β cell CHOP expression, comprising administering a nucleic acid composition comprising a Chop inhibitor moiety and a pancreatic β cell targeting moiety. The regulating can be achieved by regulating the induction of Chop inhibitor moiety and a pancreatic β cell targeting moiety. In one embodiment, the induction of an ASO may be regulated by designing an ASO expression construct that comprises a promoter operably linked to the ASO, wherein the promoter is inducible by a regulator. In some embodiments the targeting moiety may be designed such that the expression of the targeting moiety is dependent on activation by a regulator. A large number of vector and promoter systems are well known in the art. Construction of expression vectors having a promoter that is inducible by a regulator is known to one of skill in the art. Exemplary inducible promoter may be a doxycycline or a tetracycline inducible promoter. Tetracycline regulated promoters may be both tetracycline inducible or tetracycline repressible, called the tet-on and tet-off systems. The tet regulated systems rely on two components, i.e., a tetracycline-controlled regulator (also referred to as transactivator) (tTA or rtTA) and a tTA/rtTA-dependent promoter that controls expression of a downstream cDNA, in a tetracycline-dependent manner. tTA is a fusion protein containing the repressor of the Tn10 tetracycline-resistance operon of Escherichia coli and a carboxyl-terminal portion of protein 16 of herpes simplex virus (VP16). The tTA-dependent promoter consists of a minimal RNA polymerase II promoter fused to tet operator (tetO) sequences (an array of seven cognate operator sequences). This fusion converts the tet repressor into a strong transcriptional activator in eukaryotic cells. In the absence of tetracycline or its derivatives (such as doxycycline), tTA binds to the tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. In contrast, in the tet-ON system, a mutant form of tTA, termed rtTA, has been isolated using random mutagenesis. In contrast to tTA, rtTA is not functional in the absence of doxycycline but requires the presence of the ligand for transactivation. A Tamoxifen inducible system may comprise a reversible switch, that can provide reversible control over the transcription of a gene or genes that are regulated by the system. The tamoxifen/estrogen receptor regulatable system can allow spatiotemporal control of gene expression, especially when combined with the Cre/Lox recombinase system, where the Cre recombinase is fused to a mutant form of the ligand-binding domain of the human estrogen receptor resulting in a tamoxifen-dependent Cre recombinase.

In one aspect, provided herein are compositions comprising a nucleic acid sequence capable for suppressing human Chop gene expression, operably linked to a peptide. In some embodiments, the composition further comprises a targeting moiety that directs the nucleic acid sequence that inhibits Chop expression to a target in a pancreatic cell.

The nucleic acid sequence capable for suppressing or inhibiting human Chop gene expression in vivo is described in the previous paragraphs. In some embodiments, the nucleic acid sequence capable for suppressing human Chop gene expression is an RNA. In some embodiments, the nucleic acid sequence capable for suppressing human Chop gene expression is an inhibitory RNA. In some embodiments, the nucleic acid sequence capable for suppressing human Chop gene expression is an antisense oligomeric RNA. In some embodiments, the nucleic acid sequence capable for suppressing human Chop gene expression is an iRNA. In some embodiments the nucleic acid sequence capable for suppressing or inhibiting human Chop gene expression is a double stranded RNA comprising of about 22 to about 28 nucleotides, and comprises at least one overhang, wherein at least the overhang is at the 5′ end or the 3′ end.

In some embodiments, the compositions provided herein are for use in selectively inhibiting Chop in a pancreatic β cell. In some embodiments, one or more nucleic acid sequences may be incorporated in a vector. In some embodiments, the vector for expression of the recombinant protein is of a viral origin, namely a lentiviral vector or an adenoviral vector. In some embodiments, the nucleic acid encoding the recombinant nucleic acid is encoded by a lentiviral vector. In some embodiments the viral vector is an Adeno-Associated Virus (AAV) vector.

Alternatively, in one embodiment, the nucleic acid composition may be delivered inside a cell via a lipid vehicle, such as a liposome or a lipid nanoparticle. Lipid nanoparticles (LNP) may comprise a polar and or a nonpolar lipid. In some embodiments, cholesterol is present in the LNPs for efficient delivery. LNPs are 100-300 nm in diameter provide efficient means of mRNA delivery to various cell types; or can be administered. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiments, the LNP encapsulates the nucleic acid wherein the nucleic acid is inserted in a vector, such as a plasmid vector. In some embodiments, the LNP is used to deliver the nucleic acid into a subject. LNP can be used to deliver nucleic acid systemically in a subject. It can be delivered by injection. In some embodiments, the LNP comprising the nucleic acid is injected by intravenous route. In some embodiments the LNP is injected subcutaneously.

In one embodiment, provided herein is a method for treating T2D, the method comprising inhibiting C/EBP homologous protein (Chop) in pancreatic β cells by administering to the subject a composition comprising: (a) a Chop inhibiting moiety, and (b) a pancreatic β cell targeting moiety. The composition, as described in the previous paragraphs, comprising a Chop inhibiting moiety and a pancreatic β cell targeting moiety can be administered to the subject systemically. Administering the composition may be associated with the reduction of one or more conditions associated with T2D.

In one embodiment, administering the composition may be associated with reduction of pancreatic ER stress. In some embodiments, administering the composition may be associated with reduction in Ins1 and Ins2 gene expression, without disrupting normal pancreatic β cell function, or identity or normal gene expression. In some embodiments, the administration may be associated with at least 30% reduction in the expression of Ins1 and/or Ins2 gene expression. In some embodiments, the administration may be associated with at least 35%, 40%, 45%, 50% or 60% reduction in the expression of Ins1 and/or Ins2 gene expression. In some embodiments, the method is associated with reduction of UPR related gene expression in the pancreas. In some embodiments, the method is associated with reduction of UPR related gene expression in the liver. In some embodiments, the method is associated with reduction of UPR related gene expression in the pancreas and liver.

Provided herein is a method of reducing hepatic steatosis in a subject in need thereof, comprising: inhibiting C/EBP homologous protein (Chop) in pancreatic β cells by administering to the subject a composition comprising: (a) a Chop inhibiting moiety, and (b) a pancreatic β cell targeting moiety. In some embodiments, the subject is a mammal. In some embodiments, the subject is a human. In some embodiments, the subject is a non-human mammal. In some embodiments, inhibiting Chop in pancreatic β cells by administering to the subject the composition as described in the previous paragraphs may be associated with reduction in hepatomegaly.

Provided herein is a pharmaceutical composition comprising the composition as described above and a pharmaceutically acceptable excipient. Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). Acceptable carriers are physiologically acceptable to the administered patient and retain the therapeutic properties of the compounds with/in which it is administered. Acceptable carriers and their formulations are generally described in, for example, Remington' pharmaceutical Sciences (18^{th} ed. A. Gennaro, Mack Publishing Co., Easton, Pa. 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes. In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage. The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

### EXAMPLES

### Example 1. In Vivo Model for Testing Chop Deletion in Pancreatic β Cells

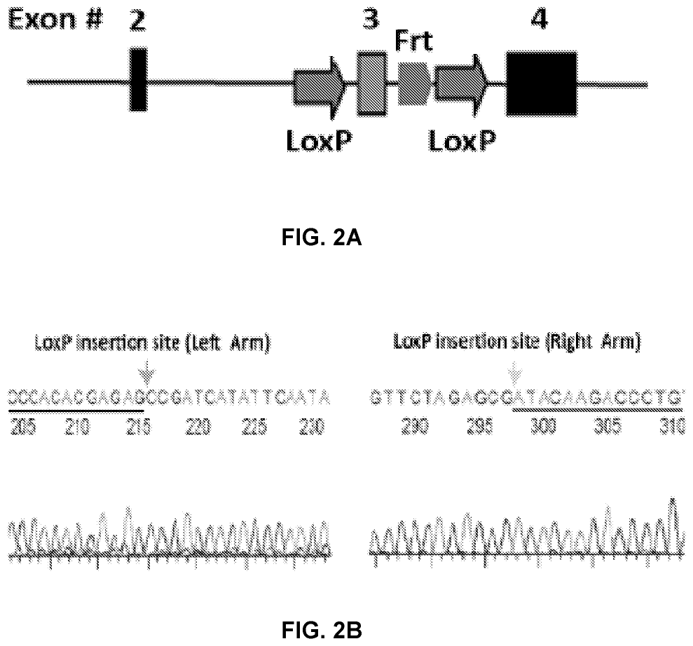

This example illustrates an exemplary mouse model for proof of principle studies of the effect of Chop deletion on pancreatic β cells. A conditional Chop deletion mouse model was generated by combining the Ddit3 floxed gene alleles (Zhou et al., 2015) with a RIP-CreER transgene. In this model, β cell-specific Chop deletion could be temporally controlled by tamoxifen (TAM) injection (referred to here as “Chop^{Δ/Δ}: RIP-Cre” or simply “Chop βKO” mice hereafter). The Chop deletion construct is depicted in FIG. 2A. The gene deletion in mouse islets was verified by nucleic acid sequencing. The Lox P sequence and the insertion sites are displayed in FIG. 2B.

Diet induced obesity (DIO) was generated in these mice by feeding the mice high fat diet. As a proof-of-principle, effect of β cell-specific Chop deletion on insulin production was tested. Fatty liver disease is associated with T2D. Whether β cell-specific Chop deletion protects fatty liver development in diet-induced obese (DIO, 45% fat in kcal) mice was also investigated, as the liver is an insulin sensitive organ and the major site for insulin action as well as its clearance. For this purpose, mice were fed HFD for 20 wks to induce hepatosteatosis, with Chop deleted at 10 wks post HFD.

### Example 2. Effect of Pancreatic β Cell Chop Deletion on Pancreatic Insulin Content and Development of Fatty Liver

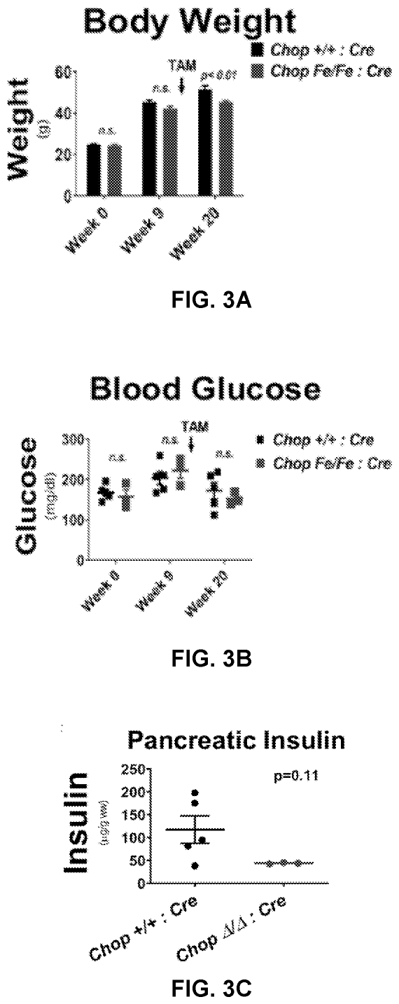

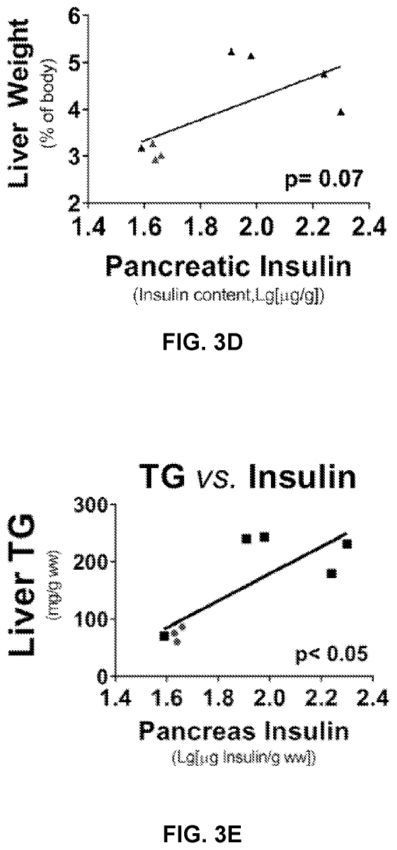

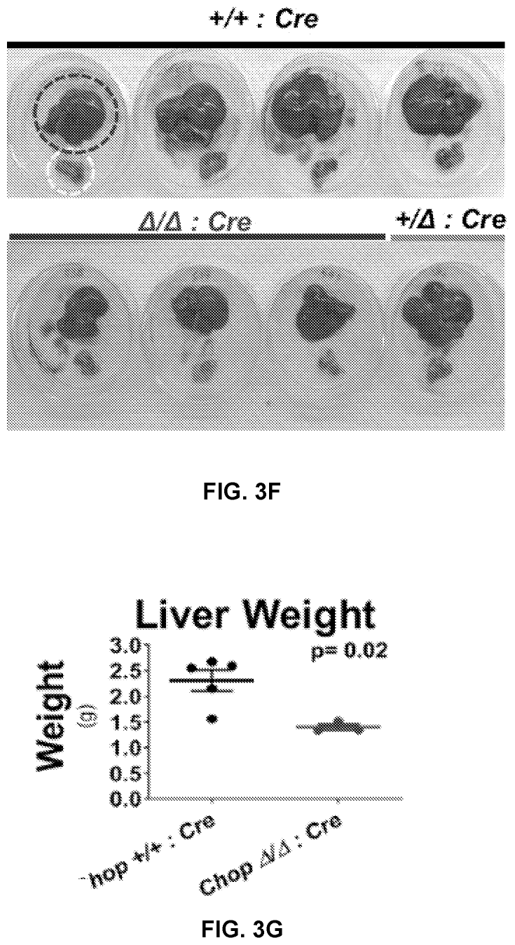

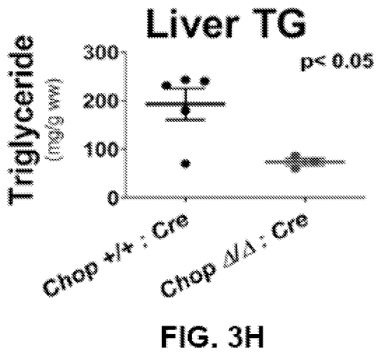

Initially, the two groups were metabolically indistinguishable as there was no significant difference in body weights or non-fasting blood glucose levels, both before or after HFD feeding (FIG. 3A and FIG. 3B). However, a moderate reduction in body weight gain was noticeable (although not significant) for Chop βKO mice, especially after TAM injection. At the end of HFD feeding, livers were dissected for visual inspection and for liver triglyceride (TG) analysis. After prolonged HFD feeding, total pancreatic insulin content was reduced 3-fold in the Chop βKO mice (FIG. 3C). Furthermore, pancreatic insulin content positively correlated with liver weight (FIG. 3D) and TG content (FIG. 3E, p<0.05 by F-test). Prolonged HFD feeding caused apparent hepatomegaly and fatty-liver for WT control mice (FIG. 3E, upper panel, indicated by the black line), as well as for the male Chop βHet mice in the litter (FIG. 3E, lower right, by the green line). In contrast, the three Chop βKO mice had normal-sized livers with a healthy appearance (FIG. 3F, lower right, left three dishes, by the red line). This phenotype was further confirmed by reduced liver weight and TG content of the Chop βKO mice (FIG. 3G & FIG. 3H, p<0.05 for both parameters, by two-tailed Student's t-test). Furthermore, in a repeat experiment using male mice (data not shown), Chop βKO DIO mice did not display any abnormalities in their gross metabolic parameters, including body weight gain, non-fasting blood glucose, food intake, or glucose tolerance compared to their Chop Het littermates (data not shown). In the same batch, Chop βKO DIO mice were moderately more sensitive to exogenous insulin administration, and the effect was not statistically significant (data not shown). In addition, Chop βKO mice had slightly decreased serum C-peptide, both before and after glucose stimulation (data not shown). An effect of Chop deletion on hepatic gluconeogenesis was ruled out, as both groups of mice showed similar glucose levels in response to pyruvate injection (data not shown).

### Example 3. Effect of Reduced Insulin Secretion on Liver Physiology

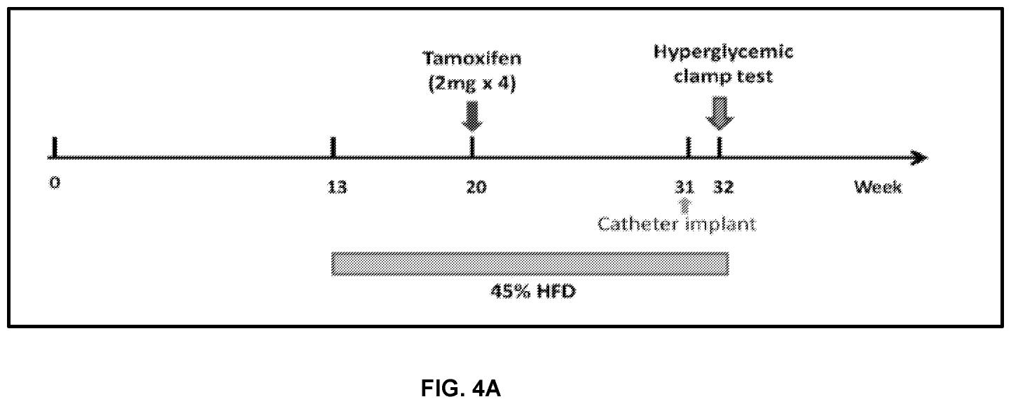

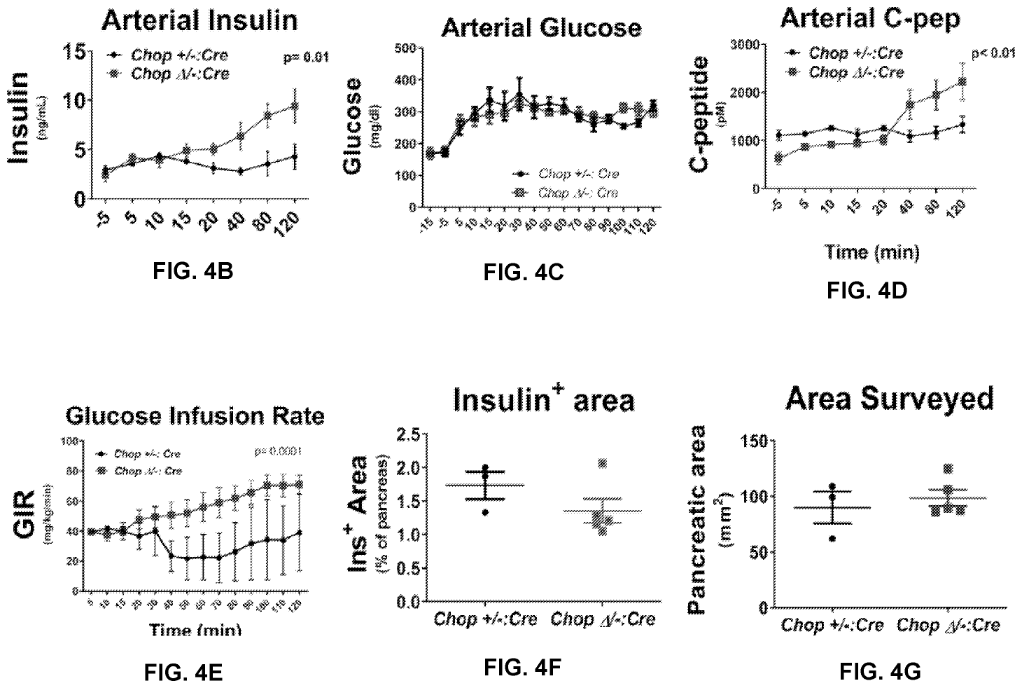

It was plausible that the reduced insulin secretion (or altered peak secretion) impacted liver physiology, therefore insulin secretion in Chop βKO mice in vivo by hyperglycemic clamp was further tested, with the age-matched Chop βHet (Chop^{+/-}: RIP-Cre) littermates as controls (both male and female mice were used). Specifically, young mice (˜7 wks old) from both groups were fed HFD for 7 wks before TAM injections (FIG. 4A). Both groups continued to be fed the HFD for another 12 wks before the clamp test. In response to hyperglycemic clamp, arterial insulin and C-peptide concentrations were significantly altered in the Chop βKO mice, characterized by observation of a cross-over ˜30 min after glucose clamping at 300 mg/dL (=16.7 mM) with a dramatic elevation in arterial insulin and C-peptide concentrations in Chop βKO mice vs. littermate controls (FIG. 4B-4D, p<0.01 for C-peptide and p=0.01 for insulin, and no difference in glucose clamping).

Furthermore, the Chop βKO mice required a significantly higher rate of glucose infusion to maintain their arterial blood glucose (FIG. 4E p=0.0001), indicating faster glucose clearance. Notably, the Chop βHet mice showed little response to high glucose stimulation, likely due to the obesity from the DIO model. The difference is not due to altered β cell mass as no difference was found in insulin-immunopositive areas for both groups (FIG. 4F & FIG. 4G). Similarly, no difference was identified for the two groups of mice in terms of their α cell distribution in islets, a cell mass, nor the relative ratio of α cells to β cells. This observation was further confirmed using the in vitro GSIS on primary Chop βKO islets, and pinpointed an islet-autonomous insulin secretion change.

### Example 4. Molecular analysis of reduced ER stress

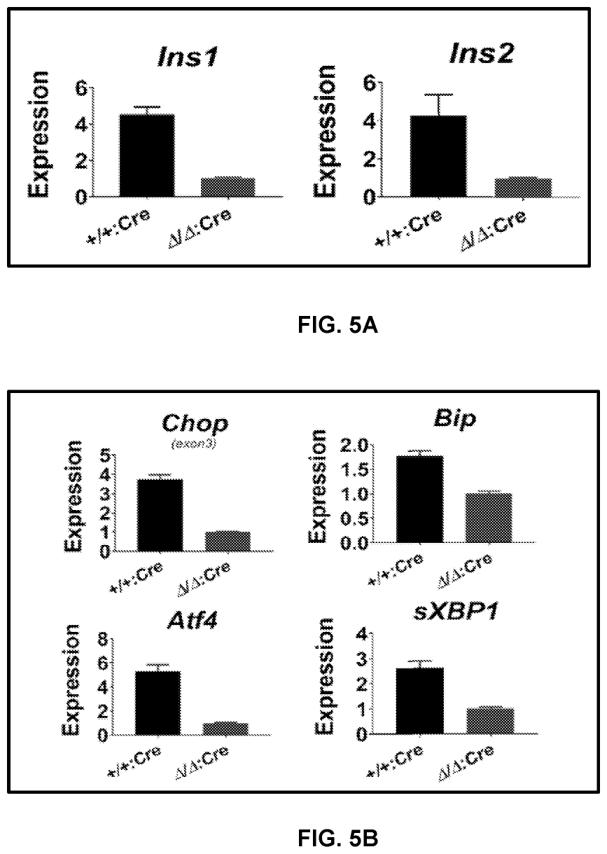

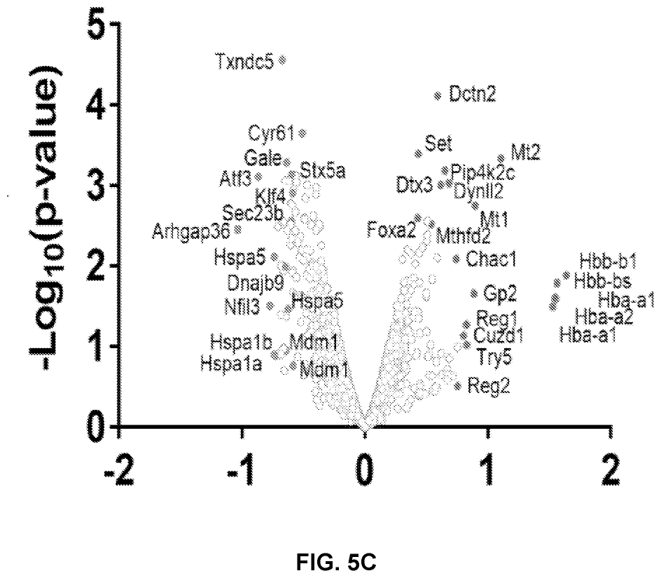

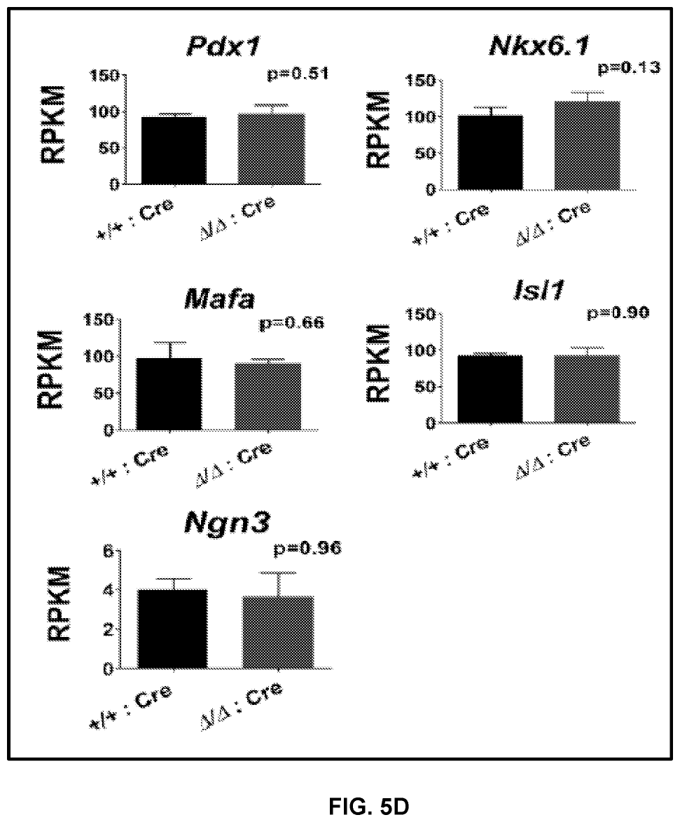

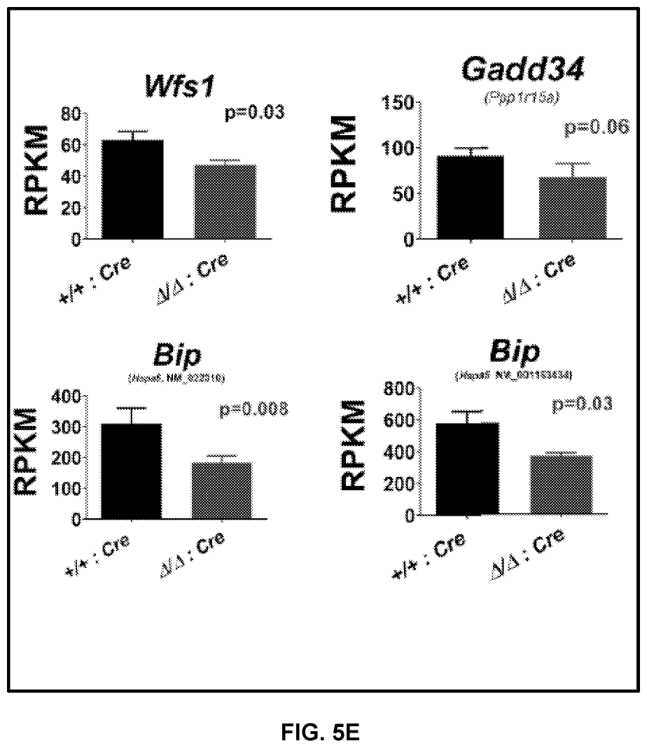

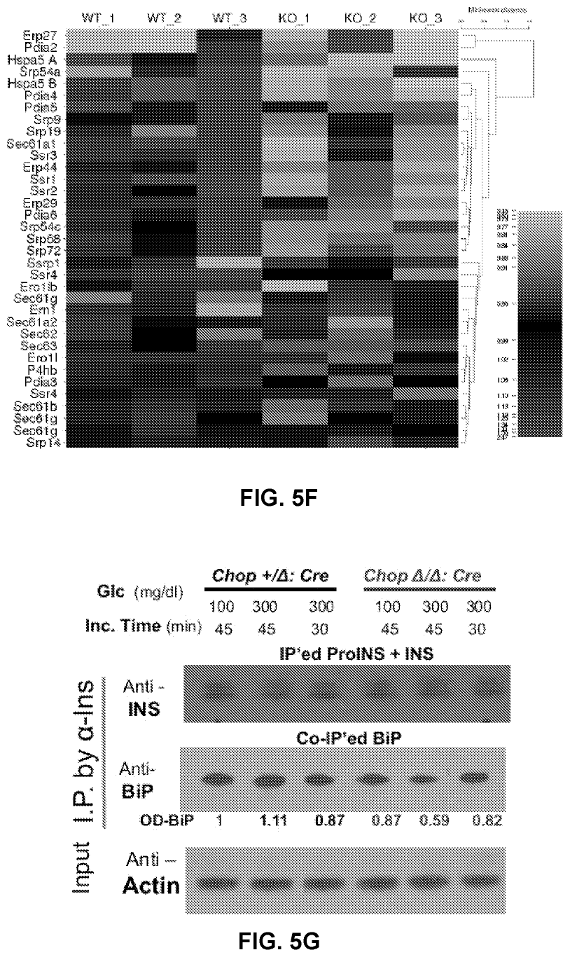

In this example, qRT-PCR analysis was performed on the RNA extracted from adult Chop βKO islets. The results revealed reduced Insulin transcript dosage associated with reduced ER stress. Two major RNA transcripts in islets, Ins1 and Ins2, were both decreased by 75% in Chop βKO islets (FIG. 5A). Transcripts encoding the other three UPR genes, Atf4, Bip and spliced XBP1 (sXBP1) were reduced in βKO islets compared to WT littermates (FIG. 5B). For further support, the same RNA materials were subjected to poly-A⁺ enriched mRNA-Seq analysis. Transcriptome profiling confirmed significant UPR reduction, represented by Atf3, Bip/Hspa5 and Dnajb9/ERdj4 on a “Volcano plot” (FIG. 5C). Chop deletion did not affect β cell identity, as key β cell transcription factors, including Pdx1, Nkx6.1, Mafa, Isl1 and Ngn3, were unaltered in Chop-deleted islets (FIG. 5D). In addition, Chop deletion did not alter mRNAs encoding Glut2, Cpe or PC1/2, further supporting the notion that Chop deletion does not alter β cell differentiation. In contrast, Wfs1, Gadd34/Ppp1r15a, and Bip/Hspa5 (encoded by two alternatively spliced isoforms, namely NM_022310 and NM_001163434), representing the downstream target genes of the IRE1α, the PERK and the ATF6α branches of the UPR, respectively, were all significantly reduced (FIG. 5E). This trend of reduced UPR transcript abundance also included a subset of mRNAs encoding important ER proteins when analyzed by heat map (FIG. 5F). Collectively, the mRNA-Seq data suggest that Chop deletion in β cells reduces ER stress, under which condition ProIns is expected to fold more efficiently in the ER. This was further tested by ProIns' interaction with BiP, a biochemical indicator for ProIns misfolding (Fan et al., 2017; Liu et al., 2005; Scheuner et al., 2005). Co-IP experiment revealed less BiP protein association with ProIns/Ins (FIG. 5G), confirming reduced ER stress in the Chop KO islets.

### Example 5. Altered ER Ca²⁺ Signaling with Chop Deletion in Islet Cells

The above results lead to the hypothesis that under physiological conditions, CHOP serves as a transcriptional hub in β cells to remodel the ER by altering expression of genes encoding structural, functional and trafficking proteins to accommodate ProIns synthesis. Conversely, removing CHOP in β cells lead to reduced ProIns synthesis burden with more efficient folding. However, an apparent mechanistic link was missing in that CHOP does not bind directly to the promoters of all of the genes affected, as we have shown previously by ChIP-Seq analysis (Han et al., 2013). The ER is the major intracellular Ca²⁺ storage organelle and cytosolic Ca²⁺ levels can be profoundly affected by it (Kaufman and Malhotra, 2014; Yong et al., 2019). Pancreatic islets exhibit glucose-dependent cytosolic Ca²⁺ oscillations that are driven by membrane electrical activity (Gilon et al., 2014; Sabourin et al., 2015)(Satin LS 2015 Mol Asp Med, Bertram 2018 Diabetes), and Insulin gene transcription is positively regulated by cytosolic Ca²⁺ (German MS 1990 JBC). It is important to determine whether the Chop deletion-dependent changes in islets are associated with altered ER Ca²⁺ signaling.

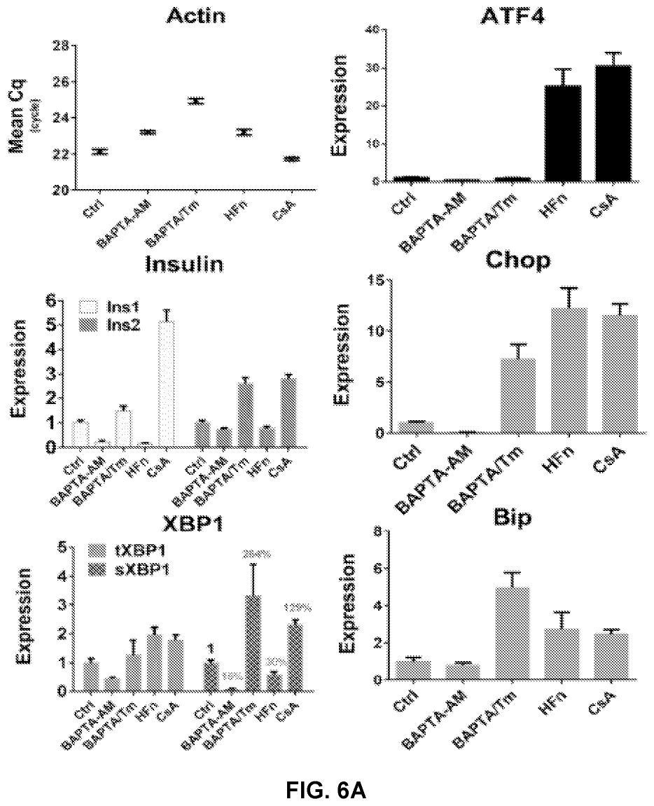

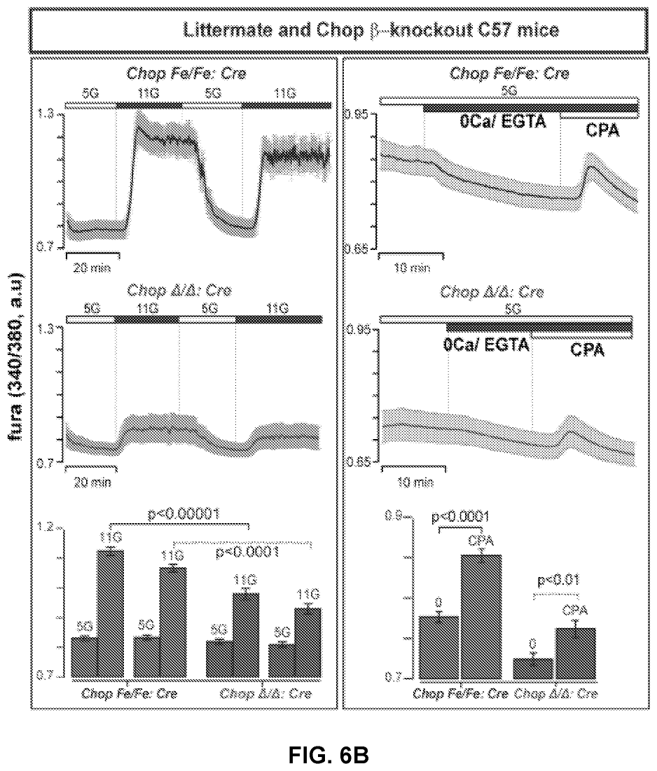

For this purpose, the effect of a membrane-permeable intracellular Ca²⁺-chelator, BAPTA-AM was tested on islets isolated from WT C57BL/6 mice. An overnight incubation with BAPTA-AM (10 μM) reduced both Ins1 and Ins2 mRNA levels (FIG. 6A, “Insulin” panel, Ctrl versus BAPTA-AM). At the same time, Chop and sXBP1 levels were also reduced by BAPTA-AM treatment, suggesting reduced UPR induction in these WT islets was due to a decreased ProIns translation burden (FIG. 6A, “Chop” and “XBP1” panel, Ctrl columns versus BAPTA-AM columns). On the other hand, tunicamycin (Tm, 0.5 μg/mL) in combination with BAPTA-AM rescued both Ins1 and Ins2 mRNA levels. Concomitantly, Chop and sXBP1 mRNAs, as well as MP mRNA were induced by Tm treatment (FIG. 6A, BAPTA-AM columns versus BAPTA/Tm columns). The positive regulation of Ins1/Ins2 transcription by ER stress, which has been previously reported (Hassler et al., 2015), was further confirmed in independent batch of WT and Chop βKO islets treated with 5 μg/mL tunicamycin (Tm). In contrast, merely inducing Chop mRNA by activating GCN2 with halofuginone (HFn, 50 nM), a potent tRNA synthetase inhibitor (Keller et al., 2012; Zhou et al., 2013) to induce eIF2α phosphorylation, reduced Ins1 and Ins2 mRNAs despite of pronounced ATF4 and Chop induction (FIG. 6A, Ctrl. Versus HFn).

Lastly, when cyclosporine A (CsA, 200 nM) was added to the islets, both Ins1 and Ins2 transcripts increased, suggesting the ER Ca²⁺ effect observed in our study is not a direct consequence of the Calcineurin/NFAT pathway (Heit et al., 2006). Insulin granule secretion from mature β cells is dependent on cytosolic Ca²⁺ oscillations and Chop βKO islets exhibited a reduction in cytosolic Ca²⁺ concentration in response to 11 mM glucose (FIGS. 6B & 6C).

### Example 6. Chop Knockdown Using GLP1-Conjugated Antisense Oligonucleotide

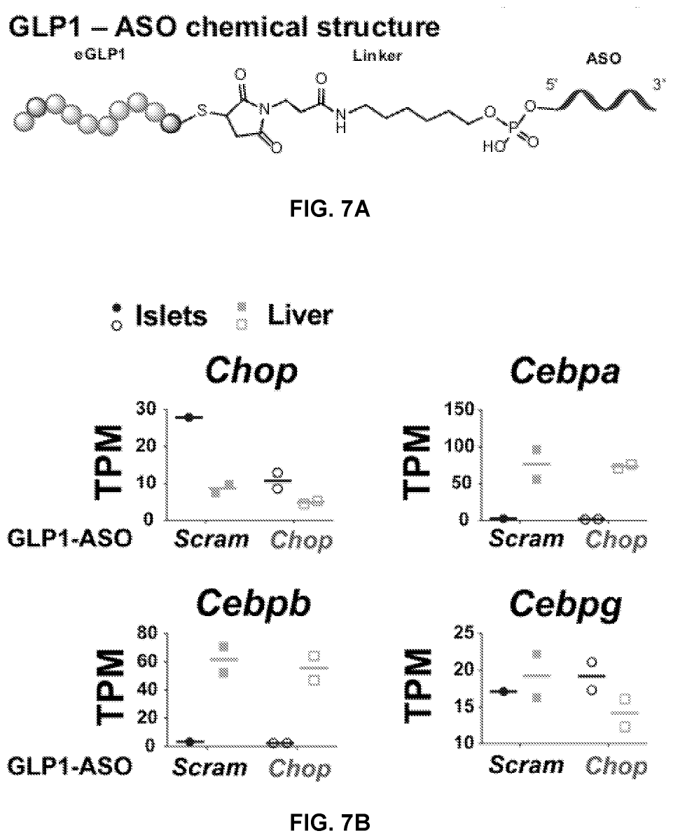

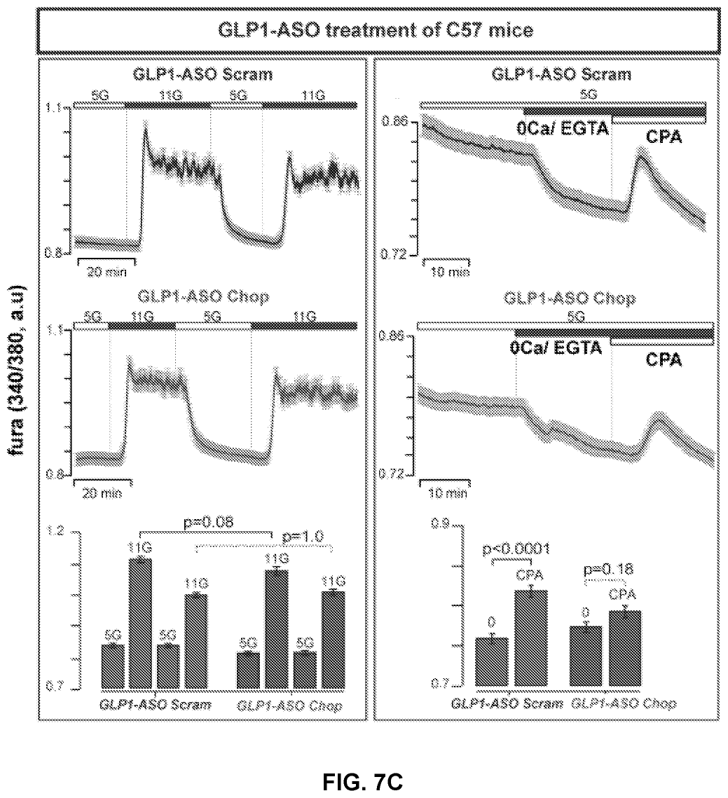

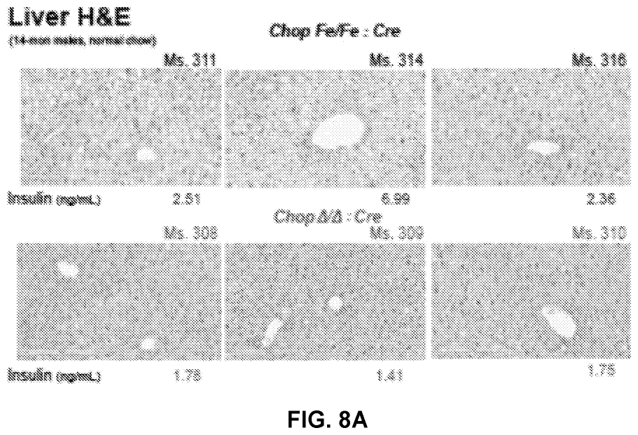

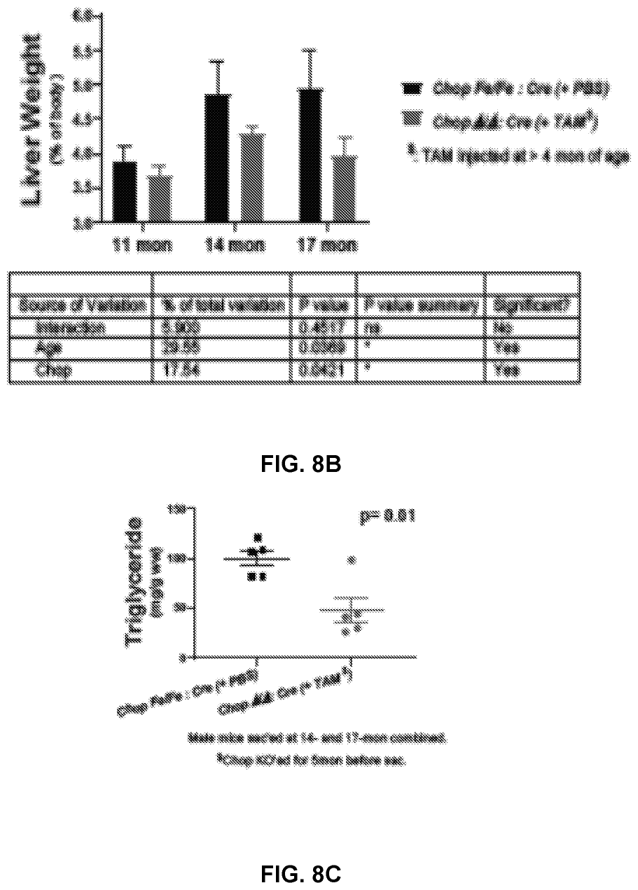

In this example, GLP1-conjugated antisense oligonucleotide (GLP1-ASO) demonstrated efficient gene knockdown in rodent islets, providing an attractive, independent strategy to mediate Chop knockdown to test the hypothesis. Mice having foxed Chop alleles (on the C57BL/6 background without CreERT) were given subcutaneous injections of GLP1-Chop-ASO as follows: Young mice of both sexes were given subcutaneous injections of GLP1-Chop-ASO or GLP-1-conjugated control ASO (Control) at 0.5 nMole/g body weight, twice over 5 days, in the back of neck. Control ASO sequence was not homologous to any known gene in the murine genome (e.g., scrambled sequence, abbreviated “scram”). GLP1-Chop-ASO administration was well tolerated by the mice and specifically reduced islet Chop transcript by >60% (FIG. 7B), with minimal effects on other C/EBP family members (i.e. Cebpa, Cebpb and Cebpg) in either islets or liver tissues, demonstrating β cell selectivity (FIG. 7B). Treatment with GLP1-Chop-ASO reduced the ER Ca2+ pool without secondarily affecting the cytosolic Ca2+levels stimulated by 11 mM glucose (compare left vs right in FIG. 7C), suggesting the change in ER Ca2+ was a primary event that immediately followed Chop knockdown. These experiments demonstrated for the first time, a proof-of-principle that GLP1-ASO strategy can be exploited to alter islet physiology and Ca2+ dynamics. These results support an ER-centric role of CHOP in pancreatic β cells.

In summary, it was proposed based on these studies that CHOP is activated by UPR signaling through the PERK branch, as a response to increased ProIns synthesis and misfolding (A. Arunagiri et al., Elife 8, e44532 (2019/06/11, 2019). In turn, CHOP serves as a transcriptional hub to maintain ER proteostasis. This process also controls Insulin transcription, partially via Ca2+ signaling, from the ER to the nucleus, although this is unlikely directly mediated by CHOP (FIG. 1, indicated by the blue arrow on right). Furthermore, ER remodeling in turn has a profound effect on ER Ca2+ that subsequently contributes to controlling the increase in cytosolic Ca2+ that occurs in response to elevated glucose (FIG. 1, as indicated by the blue arrow on left). At the same time, comparison of genetic Chop deletion model versus the GLP1-ASO mediated Chop knockdown model demonstrated that Chop deletion induced GSIS change is a long term complex event in β cells, with the ER Ca2+ pool change preceding insulin mRNA reduction and GSIS decrease. The model further illustrates that ER “stress” due to increased proinsulin synthesis is coupled to insulin secretion, mediated through an “ER Ca2+” response in β-cells. In the UPR signaling cascade, however, Perk deletion (H. P. Harding et al., Mol Cell 7, 1153; June, 2001), eIF2a phosphorylation site mutation (D. Scheuner et al., Mol Cell 7, 1165; 2001); (S. H. Back et al., Cell Metab 10, 13; July, 2009), Ire1α deletion (J. R. Hassler et al., PLoS Biol 13, e1002277 (October, 2015), sXbp1 deletion (A. H. Lee, et al., Proc Natl Acad Sci USA 108, 8885, May 24, 2011) and Atf6α deletion (F. Engin et al., Sci Transl Med 5, 211ra156, Nov. 13, 2013) all caused deleterious outcomes in β-cells and were thus unsuitable therapeutic targets. Uniquely, Chop deletion may be the only example that can safely reduce “ER stress” in β-cells, by exemplifying a “thrifty gene” providing an evolutionary advantage during famine. This hypothesis is more attractive given the finding demonstrated herein that β-cell specific Chop deletion prevented HFD-induced hepatic steatosis (e.g., FIG. 3F). Inspired by the unique phenotype of Chop-deleted β-cells, applicants discovered that a GLP1-conjugated Chop ASO could partially recapitulate “ER remodeling” characterized by a reduced ER Ca2+ pool, thereby providing a promising new therapeutic strategy for further pharmacological characterization and refinement to combat human T2D and fatty liver disease.

### Example 7. Pancreatic β-Cell Specific Chop Gene Knockout Prevents Ageing-Associated NAFLD Development in Mice

In this example, a role of Chop gene expression on age-associated NAFLD was investigated in mice, and experimentally demonstrates for the first time that suppression of Chop can have therapeutic benefit in age-associated NAFLD.

Ageing appears to be a high-risk factor for NAFLD development in humans, and in C57BL/6 mice (Bertolotti, M. et al. World J Gastroenterol 20, 14185-14204, 2014; Frith, J., D. et al., Gerontology 55, 607-613, 2009; Koehler, E. M. et al. J Hepatol 57, 1305-1311, 2012).

To test whether pancreatic β-cell specific Chop gene knockout is capable of correcting ageing-associated NAFLD in male mice, three cohorts of Chop wildtype and β-knockout (KO) littermates were sacrificed at 11-, 14-, and 17 months of age. Pancreatic β-cell specific Chop gene KO was mediated by tamoxifen injections at 5 months before sacrifice, while the control littermates received PBS as diluent controls. Ageing (with normal diet feeding) causes liver fat accumulation in mice sacrificed at 14- and 17-month of age, as evidenced by enlarged hepatocytes size by H&E staining (FIG. 7A), and by increased liver weight expressed as percentage of body weight (FIG. 7B, p<0.05 by 2-way ANOVA from “Age”). In contrast, pancreatic β-cell specific Chop gene KO leads to a reduction in fasting serum insulin concentrations that correlate with NAFLD prevention in aged mice, as reflected by normal-sized hepatocytes (FIG. 7A) and by decreased liver weight expressed as percentage of body weight (FIG. 7B, p<0.05 by 2-way ANOVA from “Chop”) comparing to their littermates control group. Furthermore, pancreatic β-cell specific Chop gene KO leads to a significantly reduced triglyceride content in aged mouse livers (FIG. 7C, p<0.01 by t-test), supporting a therapeutic benefit of NAFLD correction conferred by β-cell specific Chop gene KO.

### Example 8. Delayed, Increased Insulin Secretion as a Potential Mechanism of Action for In Vivo Insulin Reduction by β-Cell Specific Chop Gene Knockout

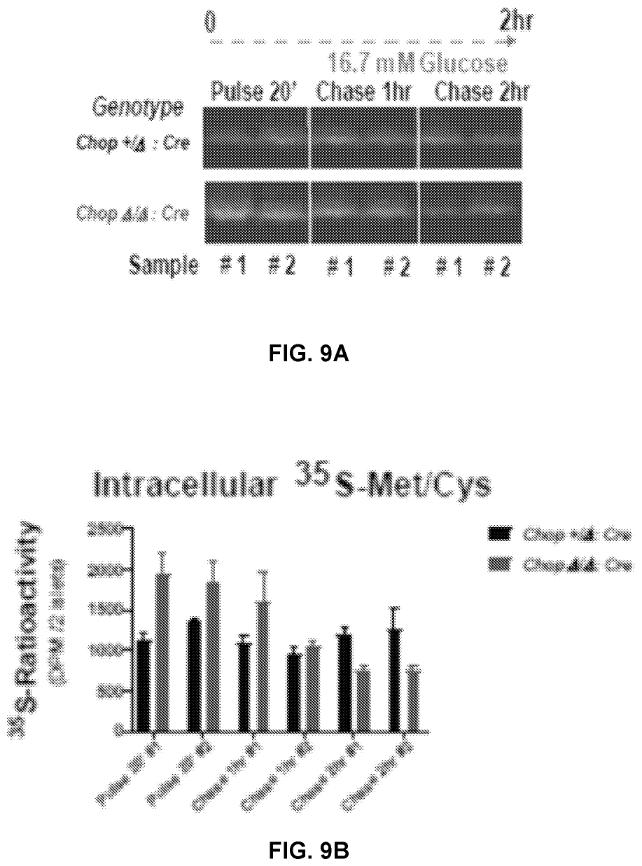

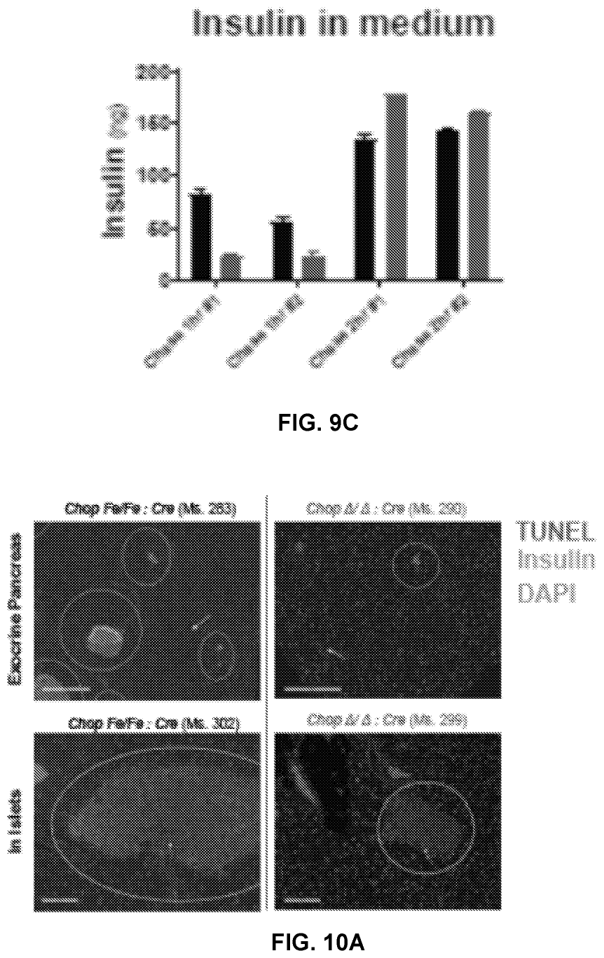

To investigate whether the altered insulin secretion in response to hyperglycemic clamp is an islet-autonomous effect, islets from a batch of lean Chop βKO mice and their age-matched littermates (Chop βHet mice) were isolated and in vitro glucose-stimulated insulin secretion (GSIS) assay was performed along with metabolic pulse-chase labeling of nascent proinsulin (ProIns) with ³⁵S-Methione+³⁵S-Cysteine in order to track the intracellular ProIns processing. Both groups of mice received TAM injections, to control for a compound effect. Results from the pulse-chase experiment suggested that the “nascent” ProIns molecules (labelled with ³⁵S-Methione/-Cysteine) was less retained in the Chop βKO islets at the end of 2 hr chase period (FIG. 9A). This observation was also confirmed by scintillation quantification (FIG. 9B) in that there is proportionally less retained ³⁵S-radioactivity in the Chop βKO islets, with the assumption that majority of reduced ³⁵S radioactivity reflects “hot insulin” secretion into the media. Newly-synthesized insulin is generally assumed to contribute proportionally more to the second phase of insulin release from β-cells. Further supporting this explanation, Chop βKO islets displayed a delayed, increased insulin secretion dynamics, which is evidenced by the cumulative insulin secretion into the conditioned media measured by ELISA assay: while there is a discernible reduction in insulin release from Chop-KO islets at 1 hr post 16.7 mM glucose stimulation (FIG. 9C), prolonged incubation of the islets in glucose for 2 hr stimulated a greater amount of insulin secretion from βKO islets (FIG. 9C) comparing to that from the βHet islets, suggesting that the peak insulin secretion is delayed but enhanced in βKO islets. In conclusion, the in vitro GSIS assay performed on isolated islets reproduced the in vivo insulin secretion pattern revealed by hyperglycemic clamp, thereby providing a potential mechanism of action for in vivo insulin reduction and hyperinsulinemia correction. It was further concluded that the delayed, increased insulin secretion from Chop KO β-cell is largely an islet-autonomous behavior.

### Example 9. No Significant Alteration in b-Cell Turnover in Chop βKO Islets

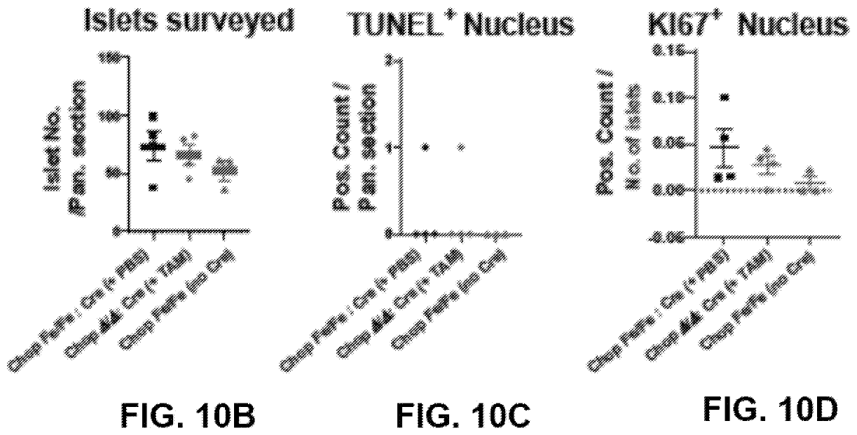

To investigate whether the in vivo effect of β-cell specific Chop gene KO is associated with altered β-cell turnover in islets, such as rate in cell proliferation (using the proliferating cell antigen Ki67 as a marker) and apoptosis (using TUNEL as a marker, pancreas sections from aged Chop βWT and Chop βKO male mice were sacrificed at 11 months of age and cells were appropriately stained with Ki67 antibody or by TUNEL staining. No changes were observed in the percentage of Ki67⁺- nor TUNEL⁺- within the pancreatic β-cell population (FIG. 10B-FIG. 10D) and is also confirmed by the histochemistry analysis shown in FIG. 10A. Consistent with the literature where a large body of evidence suggested that there is either very low or no pancreatic β-cell turnover in islets of humans and in adult mice, there is in general a very low rate of both Ki67⁺ and TUNEL⁺ cells among pancreatic β-cells in Chop βKO mice and in littermates.

1. A method of reducing hepatic steatosis in a subject in need thereof, comprising:
inhibiting C/EBP homologous protein (Chop) in pancreatic β cells by administering to the subject a composition comprising:
(a) a Chop inhibiting moiety, and
(b) a pancreatic cell targeting moiety.

2. A method of regulating C/EBP homologous protein (Chop) in pancreatic β cells, the method comprising administering a nucleic acid composition comprising:
(a) a Chop inhibiting moiety, and
(b) a pancreatic β cell targeting moiety that directs the Chop inhibiting moiety to its target in a pancreatic cell.

3. The method of claim 1 or 2, wherein a Chop inhibiting moiety is a nucleic acid.

4. The method of claim 1 or 2, wherein the Chop inhibiting moiety and the pancreatic β cell targeting moiety are operably linked.

5. The method of claim 1 or 2, wherein the nucleic acid is an RNA.

6. The method of claim 1 or 2, wherein the nucleic acid is an inhibitory RNA.

7. The method of claim 1 or 2, wherein the nucleic acid is an antisense oligomeric RNA.

8. The method of claim 1 or 2, wherein the nucleic acid is an iRNA or siRNA.

9. The method of claim 1 or 2, wherein the pancreatic β cell targeting moiety is a peptide.

10. The method of claim 9, wherein the peptide is internalized by a pancreatic cell.

11. The method of claim 9, wherein the peptide is glucagon-like peptide 1 (GLP-1), or a fragment thereof.

12. The method of claim 1 or 2, wherein the Chop inhibiting moiety is a nucleic acid editing moiety.

13. The method of claim 12, wherein the nucleic acid editing moiety is a genomic DNA editing moiety.

14. The method of claim 13, wherein the nucleic acid editing moiety comprises a nuclease.

15. The method of claim 12, wherein the nucleic acid editing moiety comprises a recombinase.

16. The method of any one of the claims 1, 2 and 12, wherein the pancreatic β cell targeting moiety comprises a guiding nucleic acid sequence.

17. The method of claim 16, wherein the Chop inhibiting moiety and/or the pancreatic β cell targeting moiety is inducible by an inducer.

18. The method of claim 17, wherein the inducer is administered ex vivo.

19. The method of claim 17, wherein the inducer is tamoxifen.

20. The method of claim 1, wherein the subject is a mammal.

21. The method of claim 1, wherein the subject is a human.

22. The method of claim 1 or 2, wherein the subject is a non-human mammal.

23. The method of any one of the claims 1-22, wherein the administering comprises administering to a subject systemically.

24. The method of any one of the claims 1-22, wherein the administering reduces or alleviates pancreatic β cell ER stress.

25. The method of any one of the claims 1-22, wherein the administering reduces total pancreatic insulin content.

26. A pharmaceutical composition comprising:
(a) (i) a nucleic acid sequence having complementarity to at least 5 consecutive nucleotides of a sequence encoding C/EBP homologous protein (Chop), (ii) a targeting moiety, wherein the nucleic acid sequence is operably linked to the targeting moiety;
(b) a pharmaceutically acceptable salt or excipient;
wherein the pharmaceutical composition is capable of suppressing human Chop gene expression.

27. The pharmaceutical composition of claim 26, for use in selectively inhibiting Chop in a pancreatic β cell.

28. The pharmaceutical composition of claim 26, wherein the nucleic acid sequence is capable of suppressing human Chop expression.

29. The pharmaceutical composition of claim 26, wherein the nucleic acid sequence capable of suppressing human Chop expression is an inhibitory RNA.

30. The pharmaceutical composition of claim 26, wherein the nucleic acid sequence capable of suppressing human Chop expression is an antisense oligomeric RNA.

31. The pharmaceutical composition of claim 26, wherein the nucleic acid sequence capable of suppressing human Chop expression is an iRNA or siRNA.

32. The pharmaceutical composition of claim 26, wherein the nucleic acid composition is targetable to a pancreatic cell.

33. The pharmaceutical composition of claim 26, wherein the peptide is glucagon-like peptide 1 (GLP-1), or a fragment thereof.

34. The pharmaceutical composition of claim 26, further comprising a linker.

35. The pharmaceutical composition of claim 34, wherein the linker is a chemical linker.

36. The pharmaceutical composition of claim 34, wherein the linker is a synthetic linker.

37. The pharmaceutical composition of claim 34, wherein the linker cross-links the nucleic acid sequence with the peptide.

38. The pharmaceutical composition of claim 26, further comprising a delivery vehicle.

39. A nucleic acid of any one of the claims 26-38, comprising about 22 to about 30 nucleotides in length.

40. The method of claim 26, wherein the peptide is a cell targeting moiety.

41. The method of claim 40, wherein the peptide has an affinity molecule on a pancreatic β cell.

42. The method of claim 40, wherein the targeting moiety is inducible by an inducer.

43. The method of claim 42, wherein the inducer is tamoxifen.

44. A cell comprising the nucleic acid composition of any one of the claims 26-38 or a part thereof.

45. A vector comprising the nucleic acid composition of any one of the claims 26-38 or a part thereof.
